SEQUENCE LISTING (3 sequences)

SEQ ID NO: 1 - protein, 5 aa, Artificial sequence
  synthetic peptide
KRRRR

SEQ ID NO: 2 - protein, 8 aa, Artificial sequence
  Epitope of ovalbumin
SIINFEKL

SEQ ID NO: 3 - DNA, 20 bp, Artificial sequence
  Oligonucleotide
cccccaccacttcccctctc

(19) United States
(12) Patent Application Publication
(10) Pub. No.: US 2007/0105765 A1
(43) Pub. Date: May 10, 2007

(54) Compositions comprising a fragment of the herpesviral protein vp22 for delivery of substances to cells

(76) Inventors: Peter Francis Joseph O'Hare, Oxted (GB); Kavitha Renga Sunassee, London (GB); Neil Douglas Brewis, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/570,487

(22) PCT Filed: Sep. 9, 2004
(86) PCT No.: PCT/GB04/03854

§ 371 Date: Jul. 19, 2006

(30) Foreign Application Priority Data
Sep. 12, 2003 (GB) 0321311.3

(51) Int. Cl. A61K 48/00; A61K 38/16; C07K 14/03

(52) U.S. Cl. 514/12; 530/350; 514/44

(57) ABSTRACT

This invention relates to aggregated compositions comprising short peptides, or sub-sequences, of full length VP22 protein for delivery of substances such as nucleic acids and proteins into cells. The invention relates to such compositions in themselves, and to methods for their manufacture and use.

### FIELD OF THE INVENTION

This invention relates to aggregated compositions for delivery of substances such as nucleic acids and proteins into cells. The invention relates to such compositions in themselves, and to methods for their manufacture and use.

### BACKGROUND OF THE INVENTION AND PRIOR ART

WO 97/05265 (Marie Curie Cancer Care: P O'Hare et al.) relates to transport proteins, in particular VP22 and homologues thereof, and to methods of delivering these proteins and any associated molecules to a target population of cells. This transport protein has applications in gene therapy and methods of targeting agents to cells where targeting at high efficiency is required.

WO 98/32866 (Marie Curie Cancer Care: P O'Hare et al.) discusses coupled polypeptides and fusion polypeptides for intracellular transport, and their preparation and use, e.g. (i) an aminoacid sequence with the transport function of herpesviral VP22 protein (or homologue, e.g. from VZV, BHV or MDV) and (ii) another protein sequence selected from (a) proteins for cell cycle control; (b) suicide proteins; (c) antigenic sequences or antigenic proteins from microbial and viral antigens and tumour antigens; (d) immunomodulating proteins and (e) therapeutic proteins. The coupled proteins can be used for intracellular delivery of protein sequences (ii), to exert the corresponding effector function in the target cell, and the fusion polypeptides can be expressed from corresponding polynucleotides, vectors and host cells.

Elliot and O'Hare (1997) Cell, vol. 88 pp. 223-233, relates to intercellular trafficking and protein delivery by a herpesvirus structural protein.

WO 00/53722 (Marie Curie Cancer Care: P O'Hare et al.) discusses certain aggregated compositions which comprise VP22 protein and oligonucleotides or polynucleotides, and use of such aggregates for delivery of substances to cells.

WO 02/20060 (Marie Curie Cancer Care: P O'Hare et al.) relates to use of certain aggregated compositions which comprise VP22 protein and oligonucleotides or polynucleotides in combination with a disaggregating agent to treat target cells by delivery of molecules to the cells.

All of these documents are hereby incorporated in their entirety by reference and made an integral part of the present disclosure.

### SUMMARY AND DESCRIPTION OF THE INVENTION

The present invention provides further aggregated compositions comprising certain short peptides, or sub-sequences, of full length VP22 protein, and oligonucleotides or polynucleotides. The invention for example provides uses of such aggregated compositions, e.g. in combination with a disaggregating agent, and also provides products comprising (a) the mentioned aggregates, and (b) a disaggregating agent, as a combined preparation for administration of the components (a) and (b) either sequentially or together, for use in therapy to treat disease or prophylactically to stimulate an immune response or to deliver desired molecules to cells, e.g. to cells in vivo or in vitro.

The VP22 protein referred to can be for example the native VP22 protein of HSV1 or HSV2.

By aggregates we mean associations of molecules forming particles for example particles of 0.1-5 microns in size e.g. of 1-3 micron in size. ‘Aggregate’ here is not intended to imply a state of denaturation or inactivity: the aggregates can usefully contain active protein and/or functionally active oligo- or polynucleotides.

According to an aspect of the invention, the mixing of oligonucleotides or polynucleotides with certain short peptides of full length VP22 protein can result in association between the nucleotide and protein fragments to form aggregates which are particularly stable, e.g. in the absence of a disaggregating agent such as actinic light, but which in the presence of a disaggregating agent such as actinic light can be particularly readily disaggregated, e.g. in target cells. A further advantage of these short peptides for forming vectosomes suitable for delivery of molecules to cells is that their small size facilitates coupling to them of other agents which it is desired to deliver to cells and subsequent delivery of the coupled molecule to cells.

Disaggregating agents such as those described in WO 02/20060 (Marie Curie Cancer Care: P O'Hare et al.) can be used to promote disaggregation of the aggregates and they can be used in the manner described in WO 02/20060.

The peptides of full length VP22 protein which can be used to form aggregates according to the present invention are peptides which are sub-fragments of the 159-301 fragment of full length VP22 protein and include proteins which correspond in sequence to amino acid residues of (a) 194-226 of full length VP22, (b) 194-220 of full length VP22, (c) 191-226 of full length VP22, and (d) 191-220 of full length VP22. The 191-220 fragment of VP22 is a further example useful for forming aggregates.

The peptides of full length VP22 which can be used to form aggregates as is described above can also be modified, for example they can be unlabelled peptides, or alternatively they can be labelled peptides. When labelled peptides are used to form aggregates it is particularly advantageous to label the VP22 peptide at the C-terminal. N-terminal labelled peptides are less preferred. In particular, N terminal labelled peptides which correspond in sequence to amino acid residues of 194-226, 191-226, and 191-220 of full length VP22 are less preferred for forming aggregates according to the invention. The label can be, for example a series of lysines and/or arginine residues, e.g. up to about eight residues, and/or a peptide, e.g. of up to about eight amino acids.

Certain peptides of full length VP22 protein which are especially preferred for making aggregates according to the invention are those which correspond in sequence to amino acid residues of: (a) 194-226 of full length VP22 and which is unlabelled (b) 194-226 of full length VP22 and which is labelled at the C terminal end of the VP22 peptide e.g. with a FITC fluorophore, (c) 191-226 of full length VP22 and which is unlabelled, and (d) 191-220 of full length VP22 and which is unlabelled. Peptides 194-226KRRRR (wherein K is lysine and R is arginine following the one letter amino acid code) and 194-226K are further useful examples for these purposes. Other examples of such peptides are 194-226KR(X6), and 194-226KR(X8) and also 194-226-X-R(X6) wherein x is an amino acid sequence such as an eight amino acid sequence, e.g. the eight amino acid epitope of ovalbumin (SIINFEKL according to the single amino acid code).

The peptides can also be modified in certain other ways, for example particular amino acids can be deleted or substituted, e.g. the cysteine of the 191-220 peptide can be replaced by an alanine.

For formation of aggregates, ratios of VP22 peptides to nucleotide are most preferred when they lead either to neutral overall charge of the aggregate molecule, or are in a higher ratio of peptide to nucleotide, leading to an overall balance of positive charge of the aggregate, e.g. in the range from about +2 to about +6, e.g. +2 to about +5, and more preferably in the range from about +2.25 to about +2.75. The relative amounts of protein and nucleotide required to achieve this can be calculated using standard methods: for example, for practical purposes a 20 mer oligodeoxynucleotide can be taken as having a −20 molecular charge, and conventional methods of protein charge calculation based on aminoacid sequence information lead to a result of +10 for the molecular charge of a VP22 159-301 polypeptide, so that a 2:1 molar ratio of peptide to nucleotide in this case leads overall to a substantially neutral balance of charge in the aggregates: and allowance can be made for different molecular charges where different polypeptide aminoacid sequences and different oligodeoxynucleotides are used.

Oligo- or polynucleotides suitable for forming part of the aggregates of the invention can preferably comprise at least 10 bases(nucleotides) and in length can range widely in size (e.g. in the range 10-50 e.g. 20 bases).

The oligonucleotides or polynucleotides contained in the aggregated composition can be DNA or RNA. When the nucleotides forming the aggregates are RNA, the ribose sugar can be 2′-O-methylated for increased nucleotide stability. In certain examples, the nucleotides can comprise phosphonate derivatives or morpholino derivatives.

The aggregates of the invention can optionally form part of a Streptavidin-biotin complex in which the oligo- or polynucleotide is labelled with biotin, e.g. at the 5′ end, and this can then be mixed with streptavidin, e.g. Streptavidin Alexa 594, which is streptavidin bound to a fluorophore molecule. Preferably, the streptavidin molecule is modified so that it can be coupled to a molecule, e.g. a drug, which it is desired to deliver to cells, e.g. so that it comprises a disulphide bond which can be used to link it to a molecule which it is desired to deliver to cells and thereby promote subsequent release of the molecule within the cell by intracellular cleavage of the disulphide bond.

Stability of the nucleotides can be increased by formation of the aggregates according to the invention. The aggregates so formed can be stable in serum, in spite of the presence of Dnases in serum. They can also be stable in high concentrations of denaturants such as urea, e.g. 7M urea.

Where the oligo- or polynucleotides contain phosphorothioate or other modified nucleotide units as mentioned above, they can be especially stable against degradation by components of serum.

The oligo- or polynucleotides contained in the aggregated compositions can contain ordinary nucleotide phosphodiester linkages. Alternatively, e.g. for achieving longer life and stability against hydrolysis, they can contain phosphorothioate linkages in place of phosphodiester linkages.

It can also be useful to label the the oligo- or polynucleotide, for example to facilitate detection and monitoring of the aggregate. The label can be at either the 5′or at the 3′ end of the synthetic nucleotide. For detection or monitoring of the aggregate any label capable of detection can be used, such as radio-label, or a fluorochrome label.

The nucleotide can be a fluorescent-labelled 20 base oligonucleotide (20-mer) containing phosphorothioate linkages. It can be labelled at the 5′ end with 5′fluorescein phosphoroamidite (Genosys), or at the 3′ end with fluorescein (Genosys), or at the 5′ end with a terminal fluoresceinyl-base (Life Technologies). Also usable is a Texas Red labelled 20 mer phosphorothioate that is labelled at the 5′ end with Texas Red (Genosys).

Aggregates according to the invention can be used to deliver their constituents into target cells.

Cells to which the aggregates can be delivered can be cells of a tissue or an organ in a mammalian subject e.g. a human subject, or they can be explanted cells, or they can be cultured cells e.g.for production of a desired protein. Cultured cells that can be used include but are not limited to CHO, COS, HeLa and Vero cells.

In certain examples, when the composition comprises a protein or peptide fused to a peptide of VP22, then the non-VP22 protein or peptide can be any which can generate an antibody or CTL immune response. Thus the compositions of the invention can be immunogenic compositions, for example they can be vaccines, e.g. DNA or protein vaccines, or both.

In certain examples, the VP22 peptide can usefully be a fusion protein in which the protein fusion partner possesses enzymatic activity. For example, a VP22 peptide-TK fusion protein, can be used in the compositions e.g. where the target cells are cancer cells e.g. neuroblastoma cells. The compositions can be delivered to target cells, and this can be followed by treatment of the target cells with ganciclovir or equivalent drugs, whereby the TK activity in the composition transported into the cell activates the ganciclovir for cell killing in per se known manner.

It can also be useful to deliver proteins of the compositions for corrective protein therapy.

It can also be useful where VP22 peptide is fused to a cell targeting peptide, such as a peptide that binds to a cell surface receptor, to facilitate cell specific targeting of the complex, e.g. the VP22 peptide can be fused to a tumour targeting molecule, such as transferrin or folate.

The oligonucleotide or polynucleotide contained in the aggregated composition according to the invention can be a substance which it is desired to deliver to a target cell.

For example, the oligonucleotide or polynucleotide can be single stranded DNA or RNA, such as a 20 mer, and it can have a base sequence that enables it, or its transcription product, to function as an antisense or ribozyme molecule in per se known manner, in effect to suppress functional expression of a chosen gene. For example the polynucleotide can be the synthetic hammerhead ribozyme, or any functional homologues or modifications thereof, which can recognise and cleave c-myb RNA, and thereby inhibit cell proliferation (Jarvis et al., J. Biol. Chem., 1996, 271, 29107-29112).

Alternatively, the oligo- or polynucleotide can be antisense, e.g. antisense to a protein which inhibits apoptosis, such as the Bcl protein, or the oligo- or polynucleotide can have the function of correcting splicing defects. The oligo- or polynucleotides can also usefully be chimeroplasts which can correct mutations.

In other examples, the oligonucleotide or polynucleotide can be single stranded DNA of appropriate sequence to enable it to bind to a specific sequence of DNA in the target cell, by forming a triple helix in per se known manner, to block transcription of the gene to which the nucleotide has bound.

In further examples, the oligonucleotide or polynucleotide can be double stranded DNA and can be of appropriate sequence to function as a binding site that binds a specific transcription factor in a target cell, thereby sequestering the transcription factor in the cell (in per se known manner) and suppressing expression of genes that depend for expression on the sequestered transcription factor.

Alternatively or additionally, the protein contained in the aggregated composition according to the invention can be a substance which it is desired to deliver to a target cell. For example, it can comprise a VP22 peptide, or a fusion protein comprising a VP22 peptide, e.g. for use as a vaccine.

The aggregated compositions according to the invention can also comprise further or other substances for delivery to target cells, such as nucleotides, proteins or peptides fused to the VP22 peptide component.

For example, the aggregated composition can comprise and deliver to a target cell linear DNA of a size sufficient to encode a gene. The delivered DNA can also comprise the necessary gene expression elements needed for its expression in the target cell.

In certain examples, the aggregated composition can comprise and deliver single stranded mRNA molecules, of size sufficient to be translated into a protein or peptide, into the cytoplasm of a target cell where the mRNA can be translated into protein or peptide.

In a further aspect of the invention, the VP22 component of the aggregate can contain a VP22 peptide and a further component, which can be either the remaining part of a fusion protein, a protein sequence of a desired functionality which it is desired to deliver within the target cell, or a nucleotide sequence which it is desired to deliver within the target cell.

The further component can be linked to the VP22 peptide by a cleavage-susceptible amino acid sequence which is susceptible to cleavage by intracellular protease within the target cell. The proteolytic site can be e.g. a site cleaved by a virus encoded protease, such as for example an HIV-encoded protease so that cleavage only occurs in virus infected cells, or alternatively the cleavage site can be one which is only cleaved by a cell-specific protease, thereby enabling delivery to a specific cell type. In this aspect of the invention, the fusion protein or coupling product can be delivered within the target cell and cleaved there by protease to release the coupling partner of the VP22 peptide, that is, the chosen protein or the nucleotide.

It can also be useful in certain examples to include a coupled protein product that is only active after cleavage of the coupled product in the target cell.

Fusogenic peptides can also be present in the aggregates according to the invention, e.g. influenza haemagluttinin for selective cell targeting and intracellular delivery.

It can also be useful to modify the oligo- or polynucleotide so that it can be coupled to a molecule which it is desired to deliver to a cell, for example through a disulphide bridge which can be reduced within the cell and thereby facilitate release of the molecule for delivery.

The aggregates can be delivered to target cells in vivo, such as cells of a tissue or an organ in a mammalian subject, e.g. a human subject. It can for example, be advantageous to deliver aggregates to cancer cells e.g. to introduce an antisense molecule which is of appropriate (per se known) sequence to target a chimeric oncogene, or to suppress a cancer gene, e.g. ras or p53, or to suppress an anti-apoptotic gene such as a member of the Bcl gene family.

The aggregates can be delivered to target cells in vivo, by for example, direct injection into target cells, such as a tumour cell mass, or they can be delivered systemically.

Alternatively, the aggregates can be formulated using per se known methods for topical delivery, e.g. for use as part of a therapy for psoriasis, eczema or skin cancer. Alternatively, the aggregates can be encapsulated into slow release capsules suitable for oral delivery using standard methods well known in the art.

The aggregates can also be associated with other delivery systems, for example they can be coupled to liposomes, such as cationic liposomes, or they can be associated with condensing agents, such as DNA condensing agents, e.g. hydrophilic polymers, e.g. protamine sulphate. They can then be delivered by e.g. direct injection into the target cells, such as tumour cells, or alternatively they can be delivered systemically, e.g. using a catheter based approach, or they can be formulated for topical delivery, nasal delivery or oral delivery.

The VP22 peptide component of the aggregates can be stored for long periods at −70 deg C., for example in a solution of PBS, or alternatively it can be lyophilised and re-constituted before use. The oligonucleotide component of the aggregates can be stored for long periods at −20 deg C. or at 4 deg C., for example in a solution of Tris buffer (pH 7.0). The VP22 peptide and oligonucleotide components can then be mixed at room temperature for at least 10 mins to enable formation of aggregates according to the invention just prior to delivery of aggregates to cells.

The aggregates can be delivered to target cells which are cells cultured in vitro, for example to CHO, COS, HeLa and Vero cells. The cultured cells containing the aggregates can be used, for example, for target validation in in-vitro testing of gene expression products.

In other embodiments, cells treated with compositions according to the invention can be explanted cells and can then be re-introduced in vivo, e.g. into a mammalian subject.

The aggregates can be substantially resistant to trypsinisation of cultured cells containing them. Therefore cells containing the aggregates in culture can be trypsinised prior to use.

In a further aspect of the invention, exposure to light such as fluorescent light can be used to promote more rapid disaggregation of the aggregates. For example, after internalisation of the aggregates, target cells in vitro can be exposed to fluorescent light, and where those cells are in vivo they can be exposed to a laser e.g. during photosurgery.

The aggregated compositions can also comprise a photosensitising molecule, e.g. fluoroscein, rhodamine, or TRITC, which can be linked to the 5′or 3′ end of the synthetic nucleotide. This can facilitate the disaggregation of the aggregates in the presence of irradiation, e.g. during phototherapy, for example, as part of a treatment for skin cancer or psoriasis. Irradiation can be achieved in vivo, for example, by introducing into a patient to be treated an endoscope comprising laser optic lines for emitting radiation. Dissociation of aggregates can also be facilitated in the absence of light by introduction of a cleavage site, such as a protease site, or a fusogenic peptide, e.g. the FLU fusion peptide.

Aggregates according to the invention can be useful as cell delivery systems for substances such as proteins or nucleotides, fused with VP22 peptide, and can enable delivery into target cells of large amounts of protein or nucleotides.

Following exposure of a cell population to such aggregates, they can be taken up by the cells and the VP22 fusion can cause transport to the cell nucleus.

Once the aggregates are taken up into a cell they have been observed in certain examples to remain within the cell for some days, and can also resist cell trypsinisation.

Also provided by the invention is a method of making such aggregates, comprising (a) mixing a VP22 peptide as mentioned above, optionally fused or covalently coupled to a protein sequence or a nucelotide for delivery to a target cell, with an oligonucleotide or polynucleotide followed by (b) incubating the mix obtained in step (a).

The invention also provides a method for transporting substances into cells, comprising contacting target cells with an aggregated composition according to the invention.

An example of the invention is described below without intent to limit its scope.

### EXAMPLE 1

This example concerns preparation of an aggregate comprising (i) a fragment of VP22, herein designated 194-226 protein, and consisting of amino acids 194-226 of the VP22 sequence of HSV2 VP22 protein, and (ii) and an oligonucleotide which is a 20 mer phosphorothioate labelled at the 5′ end with fluorescein and with a base sequence as follows:

5′ CCC CCA CCA CTT CCC CTC TC 3′.

This sequence is commercially available and is complementary to a segment of mRNA encoding an intracellular-adhesion molecule, or ICAM.

In the aggregates produced, final concentrations of protein and oligonucleotide in 50 microlitres of solution can be about 56 micromolar protein and 5 micromolar oligonucleotide.

The 194-226 protein can be synthesised by standard techniques known in the art and can be obtained from Thermo Hybaid at >95% purity (at Thermo Biosciences GmbH, Ulm, Germany).

The 194-226 protein in solution in water is used for the formation of the aggregates.

Aggregates can be produced as follows:—

25 microlitres of 20 mer phosphorothioate-linked oligonucleotide as described above (10 micromolar solution in water) labelled at the 5′ end with fluorescein is added to 25 microlitres of 194-226 protein solution in water (20 micromolar solution). The final concentration of 194-226 protein in 50 microlitres of water is about 10 micromolar and the final concentration of oligonucleotide is about 5 micromolar. The mixture is mixed and left at least 10 min at room temperature. Fifty microlitres of this mixture is then added to 450 microlitres of tissue culture medium (with or without added serum) and can be stored at about 4 deg C.

The formation of the aggregates of the invention can be monitored by using microscopy e.g. phase contrast or fluorescence microscopy, or by agarose gel electrophoresis of the aggregates.

Aggregates can be delivered to cells as follows:

Aggregates produced by the method previously described can be diluted in pre-warmed tissue culture medium and then added to COS cells and incubated for about 24 hours at a temperature of 37 deg C.

Disaggregation of the aggregates within the cells can then be promoted in a number of different ways as follows:

The COS cells are then washed in PBS and treated with trypsin in per se known manner, and then spun in a centrifuge at 2,500 rpm for 2 minutes, followed by re-suspension in PBS and re-spinning in a centrifuge. The cells are then re-suspended in 100 microlitres of serum free DMEM (Sigma). 100 microlitres of cells can then be either (a) illuminated for 30 seconds with white light using a Schott lamp, and then further incubated in normal tissue culture medium for 5 hours at 37 deg C., or (b) in the absence of illumination they can be incubated in tissue culture medium containing 10 micromols of Tamoxifen for 5 hours at 37 deg C., or (c) in the absence of illumination they can be incubated in tissue culture medium containing 100 micromols of Chloroquine for 5 hours at 37 deg C.

The cells can then be examined e.g. by confocal microscopy. Re-distribution and disaggregation of the aggregates can be observed within the cells which have been illuminated, and also those treated with either tamoxifen and or with chloroquine.

The present disclosure extends to modifications and variations of the description given herein that will be readily available to the reader skilled in the art. This disclosure incorporates the content of WO 97/05265, WO 98/32866, WO 00/53722 WO 02/20060, and Elliot and O'Hare (1997, cited above) which it is intended to treat as an integral part hereof, and this disclosure is intended to extend in particular to classes and subclasses of the products and generally to combinations and subcombinations of the features mentioned, described and referenced in the present disclosure. Documents cited herein are therefore hereby incorporated by reference in their entirety for all purposes.

1. An aggregated composition comprising (a) a sub-fragment of the 159-301 fragment of full length VP22 protein, and (b) an oligonucleotide or polynucleotide.

2. An aggregated composition according to claim 1, which further comprises a pharmaceutically acceptable excipient.

3. An aggregated composition according to claim 1, wherein the sub-fragment is a VP22 fragment selected from the group consisting of: fragments comprising amino acid residues of either (a) 194-226 of full length VP22, or (b) 191-220 of full length VP22, or (c) 191-226 of full length VP22.

4. An aggregated composition according to claim 1, wherein the sub-fragment of VP22 is labelled.

5. An aggregated composition according to claim 4, wherein the sub-fragment is either VP22 peptide 194-226KRRRR (SEQ ID NO:1) labelled at the C terminal end of VP22, or VP22 peptide 194-226K labelled at the C terminal end of VP22.

6. An aggregated composition according to claim 1, wherein the sub-fragment of VP22 is modified by deletion or substitution.

7. An aggregated composition according to claim 1, wherein the sub-fragment of VP22 is a fusion protein which also comprises a non-VP22 polypeptide sequence.

8. A method of making an aggregated composition according to claim 1 comprising (a) mixing the sub-fragment of VP22 with the oligonucleotide or polynucleotide, and (b) allowing the mixture obtained in step (a) to form aggregates, e.g. aggregates with a particle size of about 0.1 to about 5 microns, e.g. about 1 to about 3 microns, e.g. by incubating the mixture at about room temperature for at least about 10 minutes.

9. Use of an aggregated composition according to claim 1 in the manufacture of a medicament for the purpose of therapy or prophylaxis of disease.

10. Use of an aggregated composition according to claim 1 in the manufacture of a medicament to deliver desired molecules to cells in vivo.

11. Use of an aggregated composition according to claim 1 in to deliver desired molecules to cells in vitro.

12. A combined preparation comprising (a) an aggregated composition according to claim 1, and (b) a disaggregating agent for administration separately or sequentially for use in therapy to treat disease or for use prophylactically to stimulate an immune response or to deliver desired molecules to cells, e.g. in vivo or in vitro
